(19) United States
(12) Patent Application Publication
(10) Pub. No.: US 2009/0220421 A1
(43) Pub. Date: Sep. 3, 2009

(54) METHOD FOR SCREENING MTP-INHIBITING COMPOUNDS

(76) Inventors: Oliver Chevreuil, Condeissat (FR); Herve Dupont, Lyon (FR); Daniel Duerrier, Lesignieux (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/912,052

(22) PCT Filed: Apr. 12, 2006
(86) PCT No.: PCT/EP06/02647

§ 371 Date: Apr. 29, 2008

(30) Foreign Application Priority Data
Apr. 22, 2005 (FR) 0504060

(51) Int. Cl. A61K 49/00; G01N 33/53; A61P 43/00

(52) U.S. Cl. 424/9.1; 435/7.1

(57) ABSTRACT

The present invention relates to a screening method for selecting active materials that inhibit microsomal triglyceride transfer protein (MTP) and to a screening kit using the said method.

The invention relates to a screening method for selecting specific inhibitors of microsomal triglyceride transfer protein (MTP) which are efficient and have a short duration of action.

MTP (“microsomal triglyceride transfer protein”) is a transfer protein located in the reticulum of hepatocytes and enterocytes, which catalyses the assembly of biomolecules that transport triglycerides, the apoB lipoproteins.

The term apoB more particularly denotes apoprotein B48 of the intestine and apoprotein B100 of the liver, without, however, being limited thereto.

Mutations in MTP or in the B apoproteins are reflected in man by very low levels or even an absence of apoB lipoproteins. The lipoproteins containing apoB (chylomicrons, “Very Low Density Lipoproteins”=VLDL) and their metabolic residues (chylomicron remnants, “Low Density Lipoproteins”=LDL) are recognised as being a major risk factor in the development of atherosclerosis, a major cause of death in industrialised countries.

It is observed that, in individuals who are heterozygous for these mutations, levels reduced on average by a half are associated with a low cardiovascular risk (C. J. Glueck, P. S. Gartside, M. J. Mellies, P. M. Steiner, Trans. Assoc. Am. Physicians, 90, 184 (1977)).

This suggests that modulation of the secretion of triglyceride-rich lipoproteins by means of MTP antagonists and/or of secretion of apoB might be useful in the treatment of atherosclerosis and more broadly of pathologies characterised by an increase in apoB lipoproteins.

Molecules that inhibit MTP and/or the secretion of apoB might thus be useful for the treatment of diabetes-related hypertriglyceridaemia, hypercholesterolaemia and dyslipidaemia, and also for the prevention of and treating obesity.

There are already a number of examples in the literature of compounds capable of inhibiting MTP, but it is observed that since the start of the investigations on these inhibitors, none of them has become commercially available. Indeed, many development projects were stopped after the first clinical trials.

Specifically, preliminary results of clinical studies support the inhibition of MTP as a means of reducing the level of triglycerides and of cholesterol in man. However, such a therapy requires a long treatment, extending over several years. The in vivo administration to man of MTP-inhibiting compounds over such long periods might give rise to toxic effects, for instance an accumulation of lipids in the intestine and the liver, leading, for example, to hepatic steatosis.

In other words, although an in vitro primary screening test now makes it possible to identify potential MTP-inhibiting candidates, the in vivo confirmation tests show that many of these candidates are responsible for toxic effects on the liver.

To overcome this problem, it is nowadays proposed, for example, to combine MTP inhibitors with fibrates. However, this type of therapy has the drawbacks associated with the administration of two active materials (problems of dosage, compatibility, etc.).

There is consequently still a need for MTP-inhibiting active substances that induce few or no toxic side effects, in particular hepatic steatosis.

Thus, a first object of the invention consists in reducing the levels of triglycerides and cholesterol in the blood, without inducing an accumulation of lipids in the liver and in the intestine.

Another object of the invention consists in providing a screening test for selecting compounds capable of reducing hypertriglyceridaemia and cholesterolaemia with a lower risk of lipid accumulation in the liver.

Other objects will become apparent in the description of the invention discussed in further detail hereinbelow.

The present invention is based on the hypothesis that the liver resecretes the excess accumulated lipids, this excess being inherent to the inhibitory activity of the compound, since it is no longer active.

The inventors have now discovered, surprisingly, that, given that a certain potency is necessary in order to obtain a sufficient pharmacological effect, only active principles having a short duration of action and liable to give a sufficient pharmacological effect (“flash” effect) generate fewer or none of the toxic effects observed with inhibitors with a long duration of action.

In addition, since the use in man involves a chronic treatment, it is necessary for this recovery phase by the liver to take place between two administrations in order to avoid the accumulation of lipids possibly leading to the development of steatosis.

Consequently, the inventors have, for the first time, used a known qualitative test for the selection of MTP-inhibiting candidates, to determine the kinetics of action of the said candidate.

Thus, the present invention first proposes a screening method for selecting active materials that inhibit microsomal triglyceride transfer protein (MTP), comprising the steps of:
a) using a candidate compound in a test of kinetic monitoring of inhibition of a parameter associated with the inhibition of MTP (inhibition of secretion of apoB, inhibition of secretion of VLDL and the like);
b) monitoring of the kinetics of inhibition of the said parameter by the said candidate from the start of the test and for a duration of between 3 hours and 24 hours, preferably between 5 hours and 12 hours and more preferably between 6 hours and 10 hours; and
c) selection of the candidate if it has kinetics of inhibition of the said parameter characterised by:
   i) a percentage of inhibition for the said parameter of greater than or equal to 50% over a maximum duration of less than 4 hours and preferably less than 3 hours; and
   ii) a residual inhibitory activity for the said parameter of less than 20% and preferably less than 10%, beyond 10 hours, preferably beyond 8 hours and more preferably beyond 6 hours, after the start of the test.

In the screening method of the present invention, the test for kinetic monitoring of inhibition is referred to as “Test A” in the rest of the present description.

Thus, Test A defined above makes it possible to select compounds having both sufficient MTP-inhibiting activity, and an absence of residual inhibitory activity that might result in adverse effects, such as those mentioned above and in particular hepatic steatosis.

The term “sufficient MTP-inhibiting activity” means that the percentage of inhibition observed for the parameter associated with the inhibition of MTP (for example apoB or VLDL) is at least equal to 50%.

The term “residual inhibitory activity” should be understood as meaning inhibitory activity observed for the parameter associated with the inhibition of MTP (for example apoB or VLDL) of less than 20%, preferably less than 10%, and more preferably insignificant inhibitory activity versus placebo.

In the rest of the present description, the term “reversible” qualifies compounds with a short duration of action, i.e. those that have the desired “flash” effect, and “irreversible” qualifies compounds with a long duration of action.

Test A of the screening method according to the present invention allows the selection of reversible candidates.

This Test A is a kinetic monitoring of inhibition of a parameter associated with the inhibition of MTP, for example a test of inhibition of the secretion of apoprotein B (apoB), advantageously if it is an in vitro test, using hepatic or enteric cells of any type, preferably hepatic cells, such as HepG2 cells, or alternatively a test of kinetic analysis of inhibition of MTP on the secretion of very low density lipoproteins (VLDL), advantageously if it is an in vivo test. The examples that follow show, for illustrative purposes, particular modes of implementation of Test A.

Test A may be performed in vitro or in vivo. According to one preferred variant of the invention, Test A performed in vitro (noted as Aᵥᵢₜᵣₒ in the rest of the description) measures the kinetics of secretion of apoB, and Test A performed in vivo (noted as Aᵥᵢᵥₒ in the rest of the description) measures the kinetics of secretion of VLDLs.

In Test Aᵥᵢₜᵣₒ, the candidate compounds are considered as having a short duration of action if they show satisfactory reversibility, i.e. if the secretion of apoB, 24 hours after removal of the test compound, has returned to a level whose value is greater than or equal to 50% and advantageously greater than or equal to 70%, relative to a control that has not, by definition, been treated with an inhibitory compound.

According to one preferred variant of the present invention, Test A is performed in vitro, and the selected candidates are then used in one or more Tests A in vivo, in order to confirm the activity of the candidate selected after the Test A in vitro.

Test A is performed in vitro on cells in culture that are preferably hepatic cells, advantageously HepG2 cells. Test A is performed in vivo on a suitable animal model, for example rats.

In addition, Test A defined above may be combined with one or more other preselection or post-selection tests, allowing faster and/or more specific screening for the selection of MTP-inhibiting candidates with a short duration of action.

Thus, in addition to the test of reversibility of action on MTP (the kinetic Test A defined above), the screening method according to the invention may be followed or preceded by one or more qualitative tests of inhibition of the activity of MTP, for instance the test described by Wetterau and Zilversmit (Biochem. Biophys. Acta, (1986), 875-610).

This test (referred to as Test B hereinbelow) makes it possible to perform a qualitative selection of candidates with regard to their capacity to inhibit or otherwise MTP in vitro. By virtue of its nature, this test is advantageously performed before Test A defined above.

In addition, it may prove to be advantageous to use the candidate products in a test (referred to as Test C in the rest of the description) of inhibition of secretion of apoB, for example in the HepG2 human cell line, allowing analysis of the inhibitory capacity of the candidates on the secretion of apoB.

Test C is of interest, like Test B, in particular if it is used before Test A, to allow a preliminary screening of “active-inactive” type of the test compounds.

Test C is also known and commonly used in the field. Reference may be made, for example, to Dixon J. and Ginsberg H. (J. Lipid. Res., (1993), 34, 167-179) for the implementation of this test.

The screening method according to the present invention for selecting compounds with satisfactory reversibility may, according to another embodiment, also comprise one or more tests for determining whether the plasmatic metabolites of the test compounds are active or inactive.

Specifically, the problem that the present invention proposes to solve consists in selecting MTP inhibitors that have satisfactory inhibitory activity but of short duration (“flash” effect, reversible inhibitors). Consequently, it is advantageous to ensure that the plasmatic metabolites of the compounds selected or to be selected do not themselves have inhibitory activity on the same target, i.e. an IC₅₀ on MTP activity of greater than 1 μM and preferably greater than 10 μM.

Thus, Test A may also be preceded or followed, preferably followed, by one or more tests of inhibition of MTP and/or of inhibition of the secretion of VLDLs and/or of inhibition of the secretion of apoB, which are similar or even identical to the Tests B and/or C, respectively, defined above, but performed this time on the plasmatic metabolites of the candidate compounds. These tests on the metabolites will be referred to hereinbelow as Test D (metabolites/MTP inhibition test) and Test E (metabolites/apoB inhibition test).

The term “plasmatic metabolites” means the products of degradation of the candidate compounds by the live body. These metabolites are obtained according to any standard method known to those skilled in the art, for example by structural determination of the products of degradation of the candidate compounds after incubation in live cells, preferably in microsomes, followed by resynthesis of the potential metabolites.

The screening method according to the present invention may be followed by one or more tests of confirmation (post-selection) of in vivo activity. Advantageously, the screening method according to the invention will also include at least one in vivo confirmation test chosen from a qualitative test of inhibition of VLDL secretion, similar to the in vitro tests B and C defined above, and a test of the evaluation of the kinetics of inhibition of VLDL secretion, similar to the in vitro test A defined above. These two confirmation tests will be referred to as test F and test Aᵥᵢᵥₒ, respectively.

Finally, the candidates that have responded positively to the tests selected from those defined above may be subjected to a final test (Test H), in vivo, for determination of the desired pharmacological effect (reduction of triglycerides in the blood). This Test H may be of any known type, for example of the type presented in the examples that follow.

All of the tests described above, which complement/confirm test A in the screening method according to the invention are collated in the table below in chronological order, which constitutes a preferred variant of the screening method according to the invention, with mention of the test target, i.e. animal model (in particular rats) for the in vivo tests, and HepG cells, MTP proteins or any other type of liver cell for the in vitro tests:

| | | | | |
|---|---|---|---|---|
| | in vitro/ | | | |
| Test | in vivo | Substrate | Target | Description |

Preselection

| | | | | |
|---|---|---|---|---|
| B | in vitro | Candidate | MTP proteins | MTP inhibition/qualitative |
| C | in vitro | Candidate | HepG2 cells | apoB inhibition/qualitative |

Selection

| | | | | |
|---|---|---|---|---|
| Aᵥᵢₜᵣₒ | in vitro | Candidate | HepG2 cells | apoB inhibition/kinetics |

Tests on the metabolites

| | | | | |
|---|---|---|---|---|
| D | in vitro | Metabo- | MTP proteins | MTP inhibition/qualitative |
| | | lites | | |
| E | in vitro | Metabo- | HepG2 cells | apoB inhibition/qualitative |
| | | lites | | |

Post-selection -- Confirmation

| | | | | |
|---|---|---|---|---|
| F | in vivo | Candidate | - animal - | VLDL inhibition/qualitative |
| Aᵥᵢᵥₒ | in vivo | Candidate | - animal - | VLDL inhibition/kinetics |
| H | in vivo | Candidate | - animal - | Desired pharmacological |
| | | | | effect (reduction of |
| | | | | triglycerides in the blood) |

It is clearly understood that the kinetic Test A can be used as sole test, in vivo or in vitro, for the determination of MTP-inhibiting compounds with a short duration of action. It will be noted, however, that if Test Aᵥᵢₜᵣₒ is used alone in the said method, it will advantageously be followed by a Test Aᵥᵢᵥₒ of kinetic analysis of the inhibitory power of the candidate compound.

According to one preferred embodiment, the invention relates to a method for screening MTP-inhibiting compounds, comprising the selection of candidates by means of a Test Aᵥᵢₜᵣₒ as defined above, followed by the selection of the candidates that have responded positively to the Test Aᵥᵢₜᵣₒ by means of a Test Aᵥᵢᵥₒ.

According to another embodiment, the invention relates to a method for screening MTP-inhibiting compounds, comprising a preselection of candidates chosen from those that have responded positively to Test B and/or to Test C defined above, selection from the candidates derived from the preselection by means of a Test Aᵥᵢₜᵣₒ as defined above, and then selection of the candidates that have responded positively to Test Aᵥᵢₜᵣₒ by means of a Test Aᵥᵢᵥₒ.

According to yet another embodiment, the present invention relates to a screening method comprising the steps of:

a) using candidate compounds in a Test B and/or a Test C, followed by preselection of the candidates responding positively to the said Test B and/or to the said Test C;

b) using the candidate compounds derived from step a) in a Test A, and advantageously a Test Aᵥᵢₜᵣₒ, and selection of the candidates responding positively to the said Test A;

c) optional additional selection of the candidate compounds selected from step b), via analysis of the metabolites of the said candidates by means of a Test D and/or a Test E;

d) using the candidate compounds selected during steps a), b) and c) in at least one in vivo confirmation test F and/or Aᵥᵢᵥₒ, followed by selection of the candidates responding positively to Test F and/or to Test Aᵥᵢᵥₒ; and

e) confirmation of the candidates selected during the preceding step, by selection using an in vivo Test H of control of the reduction of triglycerides in the blood.

The implementation of the various tests described above is preferably performed in the order B and/or C-Aᵥᵢₜᵣₒ-D and/or E-F and/or Aᵥᵢᵥₒ-H, without this order constituting any limitation of the present invention.

The present invention also relates to the use of the screening method according to the invention comprising at least one Test Aᵥᵢₜᵣₒ, a Test Aᵥᵢᵥₒ or a combination of the two, as defined above, to determine the kinetics of a parameter associated with the inhibition of MTP by a candidate compound. The conditions for performing these tests are those described in the screening method.

Preferably, this test is used to determine whether the candidate compound has a short duration of action (“flash” effect) within the meaning of the present invention, or, on the contrary, a long duration of action.

The present invention also relates to a screening kit for selecting active materials that inhibit microsomal triglyceride transfer protein (MTP) using the screening method as defined above.

The screening kit is consequently defined as comprising all of the means for performing at least one Test A, Test Aᵥᵢₜᵣₒ and/or Test Aᵥᵢᵥₒ, defined above, and optionally one, several or all of the tests B to H defined above, in any order.

The compounds selected via the screening method according to the invention, or selected by using the kit defined above, have an entirely advantageous possible application in the treatment of hypertriglyceridaemia, hypercholesterolaemia and dyslipidaemia associated with metabolic syndrome and diabetes, and pancreatitis, but also for the prevention of and treating obesity.

Thus, the MTP inhibitors with a short duration of action selected via the screening method according to the present invention induce a significant reduction in hypertriglyceridaemia in obese Zucker rats (fatty fa/fa), an animal model of hypertriglyceridaemia, without aggravating the lipid content of the liver (steatosis), as is observed, on the contrary, with an MTP inhibitor with a long duration of action.

According to another aspect, the present invention consequently also relates to the compounds selected via the screening method according to the invention or by means of the kit according to the present invention. They can be used for the preparation of a medicament for the treatment of hypertriglyceridaemia, hypercholesterolaemia and dyslipidaemia associated with metabolic syndrome and diabetes, but also for the prevention of and treating obesity.

For purely illustrative purposes, compounds 1 and 2 described in patent application FR 2 816 940 (and WO 02/42291) are MTP-inhibiting compounds with a short duration of action.

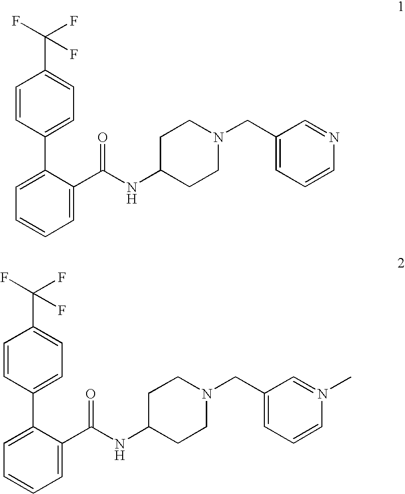

The present invention also relates to a process for the preparation or manufacture of a pharmaceutical composition, comprising a screening method according to the invention, which leads to the selection of a compound of pharmaceutical interest, and the mixing of this compound with a pharmaceutically acceptable vehicle or excipient.

The invention relates especially to such a process for the preparation or manufacture of a medicament for the treatment of lipid deregulation, such as hypertriglyceridaemia, hypercholesterolaemia and dyslipidaemia associated with metabolic syndrome and diabetes, but also for the prevention of and treating obesity.

The preparation or manufacturing process preferably comprises the implementation of the screening method according to the invention, comprising at least one Test Aᵥᵢₜᵣₒ and/or at least one Test Aᵥᵢᵥₒ defined above, to which it is possible to add one or more of the additional tests defined above, i.e. one, several or all of the Tests B to H defined above, in any order.

The present invention also relates to the pharmaceutical compositions or medicaments that may be obtained by performing the process that has just been described.

The present invention relates more generally to pharmaceutical compositions comprising a pharmaceutically effective amount of a compound selected via the screening method of the invention or via the screening kit according to the invention, in combination with one or more pharmaceutically acceptable vehicles or excipients.

These compositions may be administered orally in the form of immediate-release or controlled-release tablets, gels or granules, or in liquid form, e.g. a syrup, intravenously in the form of an injectable solution, transdermally in the form of an adhesive transdermal device, or locally in the form of a solution, cream or gel.

A solid composition for oral administration is prepared by adding to the compound selected via the screening method of the invention or via the screening kit according to the invention a filler and, where appropriate, a binder, a disintegrating agent, a lubricant, a colorant or a flavour enhancer, and by forming the mixture into a tablet, a coated tablet, a granule, a powder or a capsule.

Examples of fillers include lactose, corn starch, sucrose, glucose, sorbitol, crystalline cellulose and silicon dioxide, and examples of binders include poly(vinyl alcohol), poly(vinyl ether), ethylcellulose, methylcellulose, acacia, gum tragacanth, gelatine, Shellac, hydroxypropylcellulose, hydroxypropylmethylcellulose, calcium citrate, dextrin and pectin.

Examples of lubricants include magnesium stearate, talc, polyethylene glycol, silica and hardened plant oils. The colorant may be any colorant permitted for use in medicaments.

Examples of flavour enhancers include cocoa powder, mint in herb form, aromatic powder, mint in oil form, borneol and cinnamon powder. It should be understood that the tablet or granule may be suitably coated with sugar, gelatin or the like.

An injectable form comprising the compound, selected via the screening method of the invention or via the screening kit according to the invention as active principle is prepared, where appropriate, by mixing the said compound with a pH regulator, a buffer agent, a suspension agent, a solubiliser, a stabiliser, a tonicity agent and/or a preserving agent, and by converting the mixture into a form for intravenous, subcutaneous or intramuscular injection, according to a conventional process. Where appropriate, the injectable form obtained may be lyophilised via a conventional process.

Examples of suspension agents include methylcellulose, polysorbate 80, hydroxyethylcellulose, acacia, powdered gum tragacanth, sodium carboxymethylcellulose and polyethoxylated sorbitan monolaurate.

Examples of solubilisers include castor oil solidified with polyoxyethylene, polysorbate 80, nicotinamide, polyethoxylated sorbitan monolaurate and the ethyl ester of castor oil fatty acid.

In addition, the stabiliser includes sodium sulfite, sodium metasulfite and ether, while the preserving agent includes methyl para-hydroxybenzoate, ethyl para-hydroxybenzoate, sorbic acid, phenol, cresol and chlorocresol.

The effective administration doses and posologies of the compounds selected via the screening method of the invention or via the screening kit according to the invention, intended for the prevention or treatment of a disease, disorder or condition caused by or associated with modulation of MTP activity, depends on a large number of factors, for example on the nature of the inhibitor, the size of the patient, the desired aim of the treatment, the nature of the pathology to be treated, the specific pharmaceutical composition used and the observations and the conclusions of the treating physician.

For example, in the case of an oral administration, for example a tablet or a gel capsule, a possible suitable dosage of the compounds selected via the screening method of the invention or via the screening kit according to the invention is between about 0.1 mg/kg and about 100 mg/kg of body weight per day, preferably between about 0.5 mg/kg and about 50 mg/kg of body weight per day, more preferably between about 1 mg/kg and about 10 mg/kg of body weight per day and more preferably between about 2 mg/kg and about 5 mg/kg of body weight per day of active material.

If representative of body weights of 10 kg and 100 kg are considered in order to illustrate the oral daily dosage range that can be used and as described above, suitable dosages of the compounds selected via the screening method of the invention or via the screening kit according to the invention will be between about 1-10 mg and 1000-10 000 mg per day, preferably between about 5-50 mg and 500-5000 mg per day, more preferably between about 10.0-100.0 mg and 100.0-1000.0 mg per day and even more preferably between about 20.0-200.0 mg and about 50.0-500.0 mg per day of active material comprising a preferred compound.

These dosage ranges represent total amounts of active material per day for a given patient. The number of administrations per day at which a dose is administered may vary within wide proportions depending on pharmacokinetic and pharmacological factors, such as the half-life of the active material, which reflects its rate of catabolism and clearance, and also the minimum and optimum levels of the said active material, in blood plasma or in other bodily fluids, which are reached in the patient and which are required for therapeutic efficacy.

Many other factors should also be taken into consideration when determining the number of daily administrations and the amount of active material that should be administered in a single dosage intake. Among these other factors, and not the least of which, is the individual response of the patient to be treated.

The pharmaceutical compositions as have just been defined are preferably intended for the treatment of lipid deregulation, such as hypertriglyceridaemia, hypercholesterolaemia and dyslipidaemia associated with metabolic syndrome and diabetes, and pancreatitis, but also for the prevention of and treating obesity.

The present invention also relates to a method for the treatment of lipid deregulation, such as hypertriglyceridaemia, hypercholesterolaemia and dyslipidaemia associated with metabolic syndrome and diabetes, and pancreatitis, but also for the prevention of and treating obesity.

This method involves treating a patient in successive phases, each phase being separated from the preceding phase by a period known as the recovery period, which is sufficient to allow the liver to remove the lipids accumulated during the treatment in the preceding phase. This method advantageously uses a pharmaceutical composition or medicament according to the invention, characterised by a short duration of action within the meaning of the invention. The advantage of having compounds with a “flash” effect, thus allowing repeated periods of treatment interrupted by periods of recovery or clearance, and of adapting at will the density of the treatment as a function of the condition of the patient, the observed therapeutic effect and the tolerance, may thus be appreciated.

Under these conditions, a treatment may be undertaken using a pharmaceutical composition comprising the abovementioned compound 1 and/or 2.

The invention will now be described in greater detail with the aid of the modes of implementation taken as non-limiting examples and with reference to the drawing, in which:

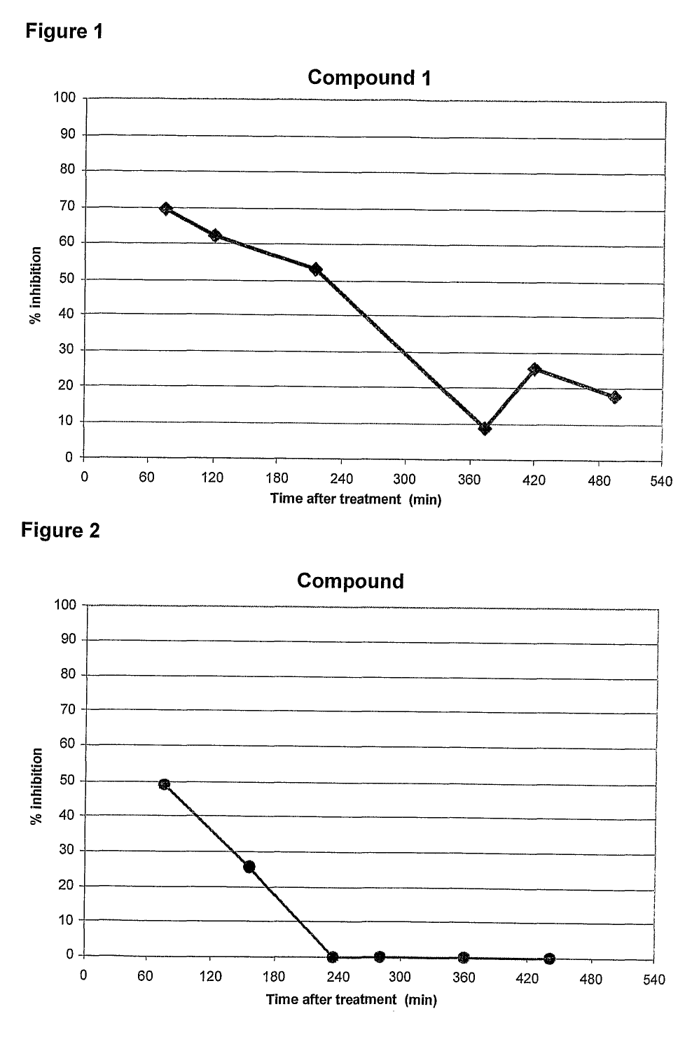

FIG. 1 is a graph with, on the x-axis, the time (minutes) after treating rats with the abovementioned compound 1, and, on the y-axis, the percentage of inhibition, illustrating the duration of action of compound 1 on the secretion of VLDL-triglycerides;

FIG. 2 is a graph with, on the x-axis, the time (minutes) after treating rats with the abovementioned compound 2, and, on the y-axis, the percentage of inhibition, illustrating the duration of action of compound 2 on the secretion of VLDL-triglycerides.

### EXAMPLES

### Test B

Qualitative Analysis of the Inhibition of Mtp Activity In Vitro

The inhibition of the activity of microsomal triglyceride transfer protein (MTP) was tested by using the following operating protocol.

The inhibition of MTP activity with a compound may be quantified by observing the inhibition of the transfer of a labelled triglyceride, from a donor particle to an acceptor particle, in the presence of MTP. The procedure for the preparation of MTP is based on the method by Wetterau and Zilversmit (Biochem. Biophys. Acta (1986) 875, 610). A few grams of golden hamster liver are taken and then rinsed several times in a 250 mM sucrose solution at 0° C. All the following steps proceed at +4° C. A homogenate at a concentration of 50% in 250 mM sucrose is prepared using a Teflon mill and then centrifuged for 10 minutes at 10 000×g at +4° C. The supernatant is then centrifuged at 105 000×g for 75 minutes at +4° C. The supernatant is discarded and the microsomal pellet is taken up in 3 ml (per g of starting liver) of Tris/HCl 150 mM pH 8.0. 1 ml aliquot fractions are stored at −80° C. until the time of use.

After thawing a fraction of microsomes (1 ml), 12 ml of refrigerated Tris/HCl 50 mM, KCl 50 mM, MgCl₂ 5 mM pH 7.4 buffer and 1.2 ml of deoxycholate (0.54% in water) are added. After incubation for 30 minutes at +4° C. with gentle agitation, the suspension is centrifuged at 105000×g for 75 minutes. The supernatant comprising the soluble MTP is dialysed against Tris/HCl 150 mM, NaCl 40 mM, EDTA 1 mM, 0.02% sodium azide pH 7.4 buffer (5 times one litre over 2-3 days). The MTP is stored at +4° C., is stable for at least 30 days and is used in unmodified form in the test.

The donor particles (liposomes) are prepared from 208 μl of L-phosphatidylcholine at a concentration of 10 mg/ml in chloroform, and 480 μl of [3H]-triolein at a concentration of 0.5 mCi/ml in toluene. After stirring, the solution is evaporated under nitrogen, taken up in 6 ml of Tris/HCl 50 mM, KCl 50 mM, MgCl₂ 5 mM pH 7.4 buffer and incubated in an ultrasound bath for 30 minutes at room temperature. The liposomes are stored at +4° C. and sonicated again for 10 minutes before each use.

The acceptor particles are biotinylated low density lipoproteins (LDL-biot). These particles are supplied by the company Amersham.

The reaction mixture is prepared in untreated ½ well white plates (Corning Costar) by addition, in the following order, of: 5 μl of HEPES 50 mM, NaCl 150 mM, BSA 0.1% (w/v), 0.05% sodium azide (w/v), pH 7.4 buffer; 5 μl of liposomes; 5 μl of LDL-biot; 5 μl of test products in DMSO 5 μl of MTP. After incubation for 18-24 hours at 37° C., the reaction is stopped by adding 100 μl of Amersham SPA (Scintillation Proximity Assay) beads coupled to streptavidin, and the radioactivity is counted using a Top Count (Packard) machine at least one hour later. The inhibition of the transfer of the triglycerides with a compound is reflected by a reduction in the transferred radioactivity. The percentage of inhibition for a given compound is determined relative to controls that do not comprise compounds in the reaction mixture.

The results are expressed in terms of the IC₅₀, i.e. the concentration that allows a 50% inhibition of MTP. These results are summarised in Table I below for compounds 1 and 2 described above.

TABLE I

| Compound | IC₅₀ (nM) |
|---|---|
| 1 | 320 |
| 2 | 65 |

### Test C

### Qualitative Analysis of the Secretion of apoB in the HepG2 Human Cell Line

The activity of a compound may be evaluated by measuring the inhibition of apoB secretion in HepG2 cells.

The HepG2 cells (ECACC-No. 85011430) are used as model in the study of the in vitro hepatic secretion of lipoproteins, as described by Dixon J. and Ginsberg H. (J. Lipid. Res., (1993), 34, 167-179).

The HepG2 cells are cultured in Dulbecco's modified Eagle's medium comprising 10% foetal calf serum (DMEM and FBS-Gibco) in 96-well plates under an atmosphere of 5% carbon dioxide for 24 hours (about 70% confluence).

The test compounds are dissolved at a concentration of 2 or 10 mM in dimethyl sulfoxide (DMSO). Serial dilutions (1:3.16) are made in DMSO and are added (1:200—Robot Multimek Beckman) to the growth medium (200 μl) and then finally incubated for 24 hours in the various wells containing the HepG2 cells.

The 24-hour culture supernatant diluted to 1:5 (phosphate-buffered saline: PBS comprising 1% bovine serum albumin) is tested according to a sandwich-ELISA method specific for human apoB.

The results, presented in Table II below for compounds 1 and 2, are expressed in terms of IC₅₀, i.e. the concentration that produces a 50% inhibition of apoB secretion in the HepG2 cells.

TABLE II

| Compound | IC₅₀ (nM) |
|---|---|
| 1 | 18.0 |
| 2 | 3.1 |

### Test Aᵥᵢₜᵣₒ

Analyse of the Kinetics (Reversibility) of Inhibition of Secretion of apoB by HepG2 Cells:

HepG2 cells are placed in contact with the products as described in the preceding paragraph and the secretion of ApoB is measured after 6 and 24 hours. The comparison is made at equipotent dose for the inhibition of secretion of apoB after 24 hours.

Next, the products are removed from the culture medium (during changes of the medium at 0, 6, 24 and 48 hours) and the secretion of ApoB is again measured to determine the kinetics for return to normal secretion. The reversibility is judged satisfactory if the secretion, 24 hours after removal of the products, has a value greater than or equal to 70% of that of the controls (blank cells without compound).

These results are collated in Table III below for compounds 1 and 2.

TABLE III

| | Secretion of apoB 24 hours after removal of the |
|---|---|
| Compound | products (% of the controls) |
| 1 | >85% |
| 2 | 85% |

### Test D

Metabolisation/Inactivation of the Product

The products are evaluated for their capacity to be inactivated rapidly in vitro. To do this, the compounds that showed satisfactory activity in vitro are tested for their rate of metabolisation in rodent liver microsomes. The incubation medium is prepared by mixing together the following constituents: NAD (1 mM), glucose 6 phosphate (5 mM), NADP (1 mM), glucose 6 phosphate dehydrogenase (1 IU/ml), bovine serum albumin (1 mg/ml), TRIS (10 mM pH=7.6), MgCl₂ (5 mM).

A concentration of 0.5 mg/ml of microsomes (Biopredic) is placed in contact with 10 μM of test product. The mixture is incubated for 1 hour at 37° C. A fraction of the preparation is taken at the initial time, to which is added an equivalent volume of ethanol (quenching of the reaction by precipitation of the proteins). The same operation is repeated after 1 hour of incubation.

The samples obtained are centrifuged at 4000 rpm for 10 minutes, and the supernatant is analysed by LC/MS/MS. The percentage of disappearance of the parent product between T0 and T60 minutes is determined by calculating the areas of the chromatographic peaks.

Potential metabolites M1 and M2 (of the compounds 1 or 2, respectively) are identified, resynthesised and evaluated for their MTP-inhibiting activity. Compounds 1 and 2 show (Table IV) metabolisation/inactivation of greater than 50% per hour into sparingly active or inactive metabolites (IC₅₀>1 μM).

TABLE IV

| | Metabolisation | |
|---|---|---|
| Compound | (%/hour) | Activity of the metabolites |
| 1 | 88 | M1, M2 inactive |
| 2 | 95 | M1, M2 inactive |

### Test F

Inhibition of Secretion of VLDL in Rats:

The inhibition of hepatic MTP by compounds 1 and 2 was evaluated from their impact on the secretion of very low density lipoproteins (VLDLs). Male Wistar rats (220-280 g) receive a standard diet and water ad libitum. On the day of the experiment, the animals (n=7) are fasted and, one hour later, are treated with the compounds (100 mg/kg orally; excipient 0.5% methylcellulose). The secretion of VLDL is measured one hour after treatment, and for 5 hours, via the triton technique (WR 1339). When it is injected into the bloodstream, triton (400 mg/kg) prevents the catabolism of the VLDLs by inhibiting their lipolysis via lipoprotein lipase and by inhibiting their uptake by tissues. Measurement of the accumulation of triglycerides (TG) and of cholesterol, which is a component of VLDLs, thus makes is it possible to reveal the secretion of VLDL by the liver.

The effect of the product on the secretion of VLDL is thus evaluated between 1 hour and 6 hours after treatment at a dose of 100 mg/kg, i.e. between 0 and 5 hours after the intravenous injection of triton. Blood samples are taken at regular intervals under gaseous anaesthesia, to assay of the triglycerides and the cholesterol. The secretion of VLDL-triglycerides and of VLDL-cholesterol is expressed in mmol/l/h (mean values). The levels of inhibition relative to controls not treated with the compounds are determined, and the results are collated in Table V.

TABLE V

| | Inhibition of secretion of | Inhibition of secretion of |
|---|---|---|
| Compound | VLDL-TG | VLDL-cholesterol |
| 1 | −56% | −45% |
| 2 | −49% | −46% |

### Test Aᵥᵢᵥₒ

Determination of the Duration of Action of MTP Inhibitors on the Secretion of VLDL

This test makes it possible to evaluate the kinetics of inhibition of MTP-inhibiting compounds on the secretion of VLDL in fatty fa/fa Zucker rats (10-12 weeks old), which is an animal model characterised by hypersecretion of VLDL. The intensity and duration of inhibition are measured via the triton technique described above over a period ranging up to 8 hours after administration of the products. Since the duration of action of a product depends both of the administered dose and on its intrinsic characteristics, it is determined at a dose of makes it possible to reach a maximum inhibition (Iₘₐₓ) of at least 50% over a significant time interval. The comparison of the profile of duration of action of two compounds that may have different potentials is thus performed at respective doses that allow similar Iₘₐₓ values to be obtained. Compounds 1 and 2 show a duration of action of less than 8 hours with an absence of significant residual inhibition between 6 and 8 hours after treatment.

The curves of inhibition as a function of time for compounds 1 and 2 are shown in FIGS. 1 and 2, respectively:

### Test H

### In Vivo Evaluation of the Antilipaemic Effect Versus the Steatogenic Effect of MTP Inhibitors

To characterise the steatogenic capacity of MTP inhibitors, a ratio between the desired pharmacological effect (reduction of triglyceridaemia or reduction of the VLDL fraction) and the potentially deleterious mechanistic effect (accumulation of triglycerides in the liver) is determined after a repeated treatment in the animal.

10-week-old fatty (fa/fa) Zucker rats are fed with a standard diet ad libitum and treated orally for 7 days (n=7-8) with compound 2 (excipient 0.5% methylcellulose). The comparison between compounds with different durations of action is made at equipotent doses for the desired pharmacological effect, i.e. reduction of the circulating triglycerides, and relates to the degree of accumulation of triglycerides in the liver relative to the control animals. The results are presented in Table VI:

TABLE VI

| | | Variation/ | | Variation/ |
|---|---|---|---|---|
| | Plasmatic | placebo | Hepatic | placebo |
| | triglycerides | (%; | triglycerides | (%; |
| Compounds | (mM) | statistical) | (mg/g tissue) | statistical) |
| Placebo | 4.29 ± 0.13 | — | 54 ± 10 | — |
| 2 | 1.40 ± 0.06 | −67%, p < 0.01 | 68 ± 15 | +28%, NS |
| (80 mg/kg/day) | | | | |

NS: not significant
Statistical: Mann-Whitney U test

1. Screening method for selecting active materials that inhibit microsomal triglyceride transfer protein (MTP), comprising the steps of:
a) using a candidate compound in a test of kinetic monitoring of a parameter associated with the inhibition of MTP;
b) monitoring of the kinetics of inhibition of the said parameter by the said candidate from the start of the test and for a duration of between 3 hours and 24 hours, preferably between 5 hours and 12 hours and more preferably between 6 hours and 10 hours; and
c) selection of the candidate if it has kinetics of inhibition of the said parameter characterised by:
i) a percentage of inhibition for the said parameter of greater than or equal to 50% over a maximum duration of less than 4 hours and preferably less than 3 hours; and
ii) a residual inhibitory activity for the said parameter of less than 20% and preferably less than 10%, beyond 10 hours, preferably beyond 8 hours and more preferably beyond 6 hours, after the start of the test.

2. Method according to claim 1, characterised in that it is performed in vitro or in vivo, preferably in vitro and more preferably in vitro and then in vivo.

3. Method according to claim 1, characterised in that the parameter associated with inhibition of MTP is the inhibition of secretion of apoprotein B (apoB), using hepatic or enteric cells of any type, such as HepG2 cells, if it is an in vitro test, and inhibition of MTP on the secretion of very low density lipoproteins (VLDL), if it is an in vivo test.

4. Method according to claim 1, characterised in that the kinetic monitoring test is preceded or followed by a test of inhibition of the activity of MTP.

5. Method according to claim 1, characterised in that the kinetic monitoring test is preceded or followed by a test of inhibition of the secretion of apoB.

6. Method according to claim 1, characterised in that it comprises one or more tests of inhibition of MTP and/or of inhibition of secretion of apoB, performed on the metabolites of the candidate compounds.

7. Method according to claim 1, characterised in that it is performed in vitro and is followed by one or more tests for confirmation of in vivo activity.

8. Method according to claim 1, characterised in that it comprises at least one in vivo confirmation test chosen from a qualitative test of inhibition of secretion of VLDL, and a test for evaluation of the kinetics of inhibition of the secretion of VLDL.

9. Method according to claim 1, comprising the steps of:
a) using candidate compounds in a Test B and/or a Test C, followed by preselection of the candidates responding positively to the said Test B and/or to the said Test C;
b) using the candidate compounds derived from step a) in a Test A, advantageously a Test Aᵥᵢₜᵣₒ, and selection of the candidates responding positively to the said Test A;
c) optional additional selection of the candidate compounds selected from step b), via analysis of the metabolites of the said candidates by means of a Test D and/or a Test E;
d) using the candidate compounds selected during steps a), b) and c) in at least one in vivo confirmation test F and/or Aᵥᵢᵥₒ, followed by selection of the candidates responding positively to Test F and/or to Test Aᵥᵢᵥₒ; and
e) confirmation of the candidates selected during the preceding step, by selection using an in vivo Test H of control of the reduction of triglycerides in the blood.

10. Use of the screening method according to claim 1, comprising at least one Test Aᵥᵢₜᵣₒ, a Test Aᵥᵢᵥₒ, or a combination thereof, to determine the kinetics of a parameter associated with the inhibition of MTP by a candidate compound.

11. Process for the preparation of a pharmaceutical composition, comprising the selection of a compound of pharmaceutical interest by means of a screening method according to claim 1, and the mixing of this compound with a pharmaceutically acceptable vehicle or excipient.

12. Process according to claim 11 for the preparation of a medicament for the treatment of lipid deregulation, such as hypertriglyceridaemia, hypercholesterolaemia and dyslipidaemia associated with metabolic syndrome and diabetes, and pancreatitis, but also for the prevention of and treating obesity.

13. Screening kit for selecting active materials that inhibit microsomal triglyceride transfer protein (MTP) using the screening method according to claim 1.
